(19) United States
(12) Patent Application Publication
(10) Pub. No.: US 2009/0269309 A1
(43) Pub. Date: Oct. 29, 2009

(54) Retinoic acid stimulates differentiation of pancreatic progenitor cells into insulin producing cells

(76) Inventors: Helena Edlund, Umea (SE); Maria Öström, Umea (SE)

Correspondence Address: SCHWEGMAN, LUNDBERG & WOESSNER, P.A., P.O. BOX 2938, MINNEAPOLIS, MN, 55402, US

(21) Appl. No.: 11/666,257

(22) PCT Filed: Oct. 28, 2005
(86) PCT No.: PCT/SE2005/001626

§ 371 Date: Jan. 18, 2008

Related U.S. Application Data
(60) Provisional application No. 60/522,690, filed on Oct. 28, 2004.

(51) Int. Cl. A61K 35/12; C12N 5/06; C12N 5/08

(52) U.S. Cl. 424/93.7; 435/377; 435/353; 435/354; 435/363; 435/366

(57) ABSTRACT

The invention relates to a method of forming pancreatic hormone-producing endocrine cells in vitro, wherein pancreatic stem cells are treated with one or more retinoids and/or retinoic acid in vitro and cultured. Especially the dorsal pancreatic bud is used and to the cells obtained by the method. Further, it relates to the use of the pancreatic hormone-producing endocrine cells, for the production of a pharmaceutical composition for transplantation and/or treatment of type 1 and type 2 diabetes. It also relates to a method for treatment of type 1 and type 2 diabetes by administrating the hormone-producing endocrine cells to individuals in need thereof. The invention also regards the use of one or more retinoids and/or retinoic acid for the differentiation, in vitro of pancreatic stem cells into pancreatic hormone-producing endocrine cells, such as glucagon producing cells, and in particular, insulin producing β-cells.

The present invention relates to a method of in vitro production of pancreatic hormone-producing endocrine cells, such as glucagon producing cells, and in particular, insulin producing β-cells.

Further, it relates to the use of the pancreatic hormone-producing endocrine cells, for the production of a pharmaceutical composition for transplantation and/or treatment of type 1 and type 2 diabetes.

It also relates to a method for treatment of type 1 and type 2 diabetes by administrating the hormone-producing endocrine cells to individuals in need thereof.

The invention further regards the use of one or more retinoids and/or retinoic acid for the in vitro differentiation of pancreatic stem cells into pancreatic hormone-producing endocrine cells.

### BACKGROUND OF THE INVENTION

A stem cell is a cell type that has a unique capacity to renew itself and to give rise to specialised or differentiated cells. Although most cells of the body, such as liver cells or skin cells, are committed to conduct a specific function, a stem cell is uncommitted, until it receives a signal to develop into a specialised cell type. The proliferative capacity of stem cells, combined with their ability to become specialised makes them unique. Embryonic stem cells are derived from the preimplanted fertilized oocyte, i.e. blastocyst. According to many national laws in Europe and other countries, a fertilized oocyte is not regarded as an embryo before implantation in the uterus i.e. about 14 days after fertilization, and such cells are therefore referred to as blastocyst-derived stem cells.

There are different types of stem cells. Totipotent stemcells can differentiate into the widest variety of cells. They contain all the genetic information needed to create all the cells in the human body in addition to the placenta. Pluripotent stem cells can give rise to all the different cell types in the human body but do not contain the genetic information to make a placenta and cannot develop into an embryo. Multipotent stem cells are cells that can divide and grow into several differentiated cell types within a specific type of tissue or organ. Progenitor cells are the least flexible stem cells. They are also called precursor or pre-specialised cells and are more differentiated than multipotent stem cells.

The pancreatic stem cells present in the early pancreatic anlagen or rudiments proliferate and eventually give rise to all pancreatic stem cell types (Edlund 2002). Notch signalling controls pancreatic cell differentiation via lateral inhibition such that activated, intracellular Notch appears to block differentiation and thus maintain cells in the pancreatic stem cell state (Apelqvist et al 1999, Hart et al., 2003). In addition, persistent expression of Fgf10 in pancreatic stem cells also inhibits pancreatic cell differentiation, while stimulating pancreatic stem cell proliferation (Hart et al., 2003). One of the effects exerted by Fgf10 under these conditions appears to be to maintain Notch activation suggesting that Fgf10 and Notch-signalling co-operate in blocking differentiation (Hart et al., 2003). Relatively little, however, is known about factors stimulating pancreatic stem cell differentiation.

### PRIOR ART

Retinoic acid (RA) has been shown to play a central role in the specification and differentiation of neuronal precursors (Diez del Corral et al., 2003; Novitch et al., 2003).

WO 01/83712 describes a method of forming vertebrate pancreas in vitro, wherein a whole or a part of a vegetal pole side region of a vertebrate blastula or a gastrula is treated with retinoic acid in vitro, then cultured.

WO 01/92480 describes another method of forming vertebrate pancreas in vitro, wherein a piece of presumptive ectoderm of a vertebrate blastula or gastrula is treated with activin and retinoic acid in vitro, then cultured.

These two methods make use of the vertebrate blastula or gastrula that are at hand about 5 days after fossilisation. These are pluripoten and the cells of the oocyte have not differentiated to an extent that pancreatic tissue has been developed.

It has now turned out that RA and retinoids may be used for specification and differentiation of pancreatic stem cells into pancreatic cells that produce insulin and glucagon.

### SUMMARY OF THE INVENTION

The invention relates to a method of forming pancreatic hormone-producing endocrine cells in vitro, wherein pancreatic stem cells are treated with one or more retinoids and/or retinoic acid in vitro and cultured. Especially the dorsal pancreatic bud is used and to the cells obtained by the method.

Further, it relates to the use of the pancreatic hormone-producing endocrine cells, for the production of a pharmaceutical composition for transplantation and/or treatment of type 1 and type 2 diabetes. It also relates to a method for treatment of type 1 and type 2 diabetes by administrating the hormone-producing endocrine cells to individuals in need thereof.

The invention also regards the use of one or more retinoids and/or retinoic acid for the differentiation, in vitro of pancreatic stem cells into pancreatic hormone-producing endocrine cells, such as glucagon producing cells, and in particular, insulin producing β-cells.

### BRIEF DESCRIPTION OF FIGURES

FIG. 1A. RA-stimulates differentiation of pancreatic stem cells into hormone-producing endocrine cells such as glucagon, and in particular, insulin cells. Immunostaining of explants cultured in medium alone (N2) or media supplemented with 25 nm RA (25 nM RA) using anti-insulin (green) and anti-glucagon (red) antibodies. DAPI (blue) stains all nuclei and was used to visualise all cells in the explant.

FIG. 1B. Quantitative summary of effects of RA on stimulation of endocrine cell differentiation from pancreatic stem cells. A>3-fold increase in the number of insulin positive cells/total number of cells was observed in response to addition of 25 nM RA: No significant effect was observed with respect to the number of glucagon cells. N=6 ** p-value<0.01.

FIG. 2. A RA-stimulated increase endocrine cell differentiation is preceded by an increase in the specification of proendocrine cells. Immunostaining of explants cultured in medium alone (N2) or media supplemented with 25 nm RA (25 nM RA) for 2 and 4 days using anti-insulin (green), anti-glucagon (red) antibodies and antibodies against the proendocrine marker ngn3 (red). DAPI (blue) stains all nuclei and was used to visualise all cells in the explant.

FIG. 2. B Quantitative summary of the effects of FIG. 2 A. The effects of RA on stimulation of ngn3-positive cells from pancreatic stem cells is shown. A five-fold increase in the number of ngn3-positive cells/total number of cells was observed in response to addition of 25 nM RA.

FIG. 3. Whole-mount immunohistochemistry of an early e10 embryo, using an IPF1 specific antibody.

IPF1/PDX1 is expressed in the developing pancreas from e 8,5 until e11. It is then down regulated but reappears later in differentiated β-cells. At the stage shown here, IPF1 stains the two pancreatic domains, denoted the ventral and the dorsal bud. The encircled dorsal bud can be identified in e10 embryos and dissected out.

### DETAILED DESCRIPTION

One first aspect of the invention relates to a method of forming pancreatic hormone-producing endocrine cells in vitro, wherein pancreatic stem cells are treated with one or more retinoids and/or retinoic acid in vitro and cultured.

By pancreatic stem cells we understand multipotent, organ specific stem cells that have an ability to differentiate to a all pancreatic cell types and among them hormone producing endocrine cells.

The pancreatic stem cells are preferably chosen from the dorsal pancreatic bud. The dorsal pancreas is an embryonic outpocketing or bud from the endodermal lining of the gut on the dorsal wall cephalad to the level of the hepatic diverticulum, which forms most of the pancreas and its main duct.

The pancreatic stem cells may be obtained from the fertilized oocyte when pancreatic tissue has started to develop. This could possibly take place from day 6 but more certainly form day 8 after fertilization. The dorsal buds should preferably be obtained before most pancreatic cells have been terminally specified i.e. before 14 days after fertilization. Especially the fertilized cells from which the dorsal pancreatic buds are obtained should not be older than 13 days. Further, the pancreatic stem cells may preferably be obtained from a fertilized oocyte before implantation in the uterus has taken place i.e. about 14 days after fertilisation. Thus, the cells are preimplanted fertilized oocyte, i.e. blastocysts and are not regarded as being obtained from an embryo according to most European legislation. Preferably the pancreatic stem cells are obtained from eight to twelve-day-old embryos, especially from 10 days old oocytes.

The pancreatic stem cells may be derived from vertebrates such as rodents such as mice, rats; pigs; monkeys and humans.

The cells may be obtained from animals by sacrificing pregnant females after the appearance of a vaginal plug and collecting the oocytes, preembryos or embryos.

They may also be obtained an artificially fertilized egg from a vertebrate such as humans, of course after consent from the couple involved, by isolating embryonic stem cells from blastocytes and stimulate theses to produce pancreatic stem cells.

Retinoic acid and retinoids may be used. Retinoids are defined as natural or synthetic analogues of retinoic acid—an active metabolite of vitamin A—or, more generally, as ligands of two classes of nuclear receptors that mediate the biological activities of retinoic acid, that is retinoic acid receptors (RARs) and retinoid X receptors (RXRs) and the subtypes α, β and γ of these two receptor families. These ligands can be agonists or antagonists to the RARs and RXRs and need not be structurally related to retinoic acid. Atypic retinoids may also be used. They have a retinoid-like structure but, in some cases, do not exhibit retinoid activities. Instead, they have retinoid nuclear receptor-independent activities. The skilled person can establish if a retinoid is useful according to the invention by establishing if glucagon and/or insulin producing cells are obtained. This may be done by immunohistochemistry using antibodies specific for glucagon and insulin as described in Example 2.

Examples of retinoids are human endogenous retinoids all-trans-retinoic acid, and 9-cis-retinoic acid; etretinate, docosahexaennoic acid and analogues thereof. Further useful examples may be found in Kagechika et al., 2005.

The treatment with retinoids and/or retinoic acid is conducted at a concentration of 1-500 nM, such as 10-200 nM, especially at 20-100 nM. This may take place for 2 to 10 days, such as 3 to 8 days, especially 5-7 days at 30° C.-40° C., preferably at 35° C.-39° C. most preferred at 37° C.

According to a second aspect the invention regards pancreatic hormone-producing endocrine cells. These cells may be obtained by the method of forming pancreatic hormone-producing endocrine cells in vitro wherein pancreatic stem cells are treated with one or more retinoids and/or retinoic acid in vitro and cultured.

A third aspect of the invention regards the use of pancreatic hormone-producing endocrine cells, obtainable by treating pancreatic stem cells with one or more retinoids and/or retinoic acid in vitro and culturing them, for the production of a pharmaceutical composition for transplantation and/or treatment of type 1 and type 2 diabetes.

Such as composition of pancreatic stem cells may comprise any excipient or additive that are compatible with the cells. By the expression “comprising” we understand including but not limited to. Thus, other non-mentioned carriers or additives may be present.

A fourth aspect of the invention relates to a method for treatment of type 1 and type 2 diabetes wherein pancreatic hormone-producing endocrine cells, obtainable by treating pancreatic stem cells with one or more retinoids and/or retinoic acid in vitro and culturing them, are transplanted into a individual in need thereof.

According to a fifth aspect the invention relates to the use of one or more retinoids and/or retinoic acid for the differentiation, in vitro of pancreatic stem cells into pancreatic hormone-producing endocrine cells, such as glucagon producing cells, and in particular, insulin producing β-cells.

All detailed descriptions herein of specific useful items are applicable to all the aspects of the invention. Thus, for examp-le the specification of retinoids and retinoic acid, mentioned in relation to the first aspect of the invention, are equally relevant for the any of the other aspects of the invention.

The invention will now be further elucidated by way of the following non limiting examples. All references cited herein are incorporated by reference.

### Example 1

### Dissection and Culturing of Explants

From mating between C57BL/6 (wild-type) mice, pregnant females were sacrificed ten days after the appearance of a vaginal plug, and embryos collected. The ten-day-old embryos were dissected in L15 medium (Gibco) using forceps and tungsten needles. The L15 medium is the 11415, 1× Liquid mg/L comprising

| | | mg/L |
|---|---|---|
| INORGANIC SALTS: | | |
| CACI₂ (anhyd) | | — |
| CACI₂ × 2H₂O | | 185.00 |
| KCI | | 400.00 |
| KH₂PO₄ | | 60.00 |
| MgCI₂ (anhyd) | | — |
| MgCI₂ × 6H₂O | 200.00 | |
| MgSO₄ (anhyd) | | — |
| MgSO₄ × 7H₂O | | 200.00 |
| NaCI | | 8000.00 |
| Na₂HPO₄ | | 190.00 |
| OTHER COMPONENTS: | | |
| D(+) Galactose | | 900.00 |
| Phenol Red | | 10.00 |
| Sodium Pyruvate | | 550.00 |
| AMINO ACIDS: | | |
| L-Alanine | | 225.00 |
| L-Arginine (freebase) | | 500.00 |
| L-Asparagine | 250.00 | |
| L-Cystine (freebase) | | 120.00 |
| L-Glutamine | 300.00 | |
| L-Alanyl-L-Glutamine | | — |
| Glycine | | 200.00 |
| L-Histidine (freebase) | | 250.00 |
| L-Isoleucine | 250.00 | |
| L-Leucine | | 125.00 |
| L-Lysine (freebase) | | 75.00 |
| L-Methionine | 75.00 | |
| L-Phenylalanine | | 125.00 |
| L-Serine | | 200.00 |
| L-Threonine | 300.00 | |
| L-Tryptophan | 20.00 | |
| L-Tyrosine | | 300.00 |
| L-Valine | | 100.00 |
| VITAMINS: | | |
| D-Ca Pantothenate | | 1.00 |
| Choline Chloride | | 1.00 |
| Folic Acid | | 1.00 |
| i-Inositol | | 2.00 |
| Niacinamide* | 1.00 | |
| Pyridoxal HCI | — | |
| Pyridoxine HCI | | 1.00 |
| Flavin Monoucleotide | | 0.10 |
| Thiamine Monophosphate HCI | | 1.00 |

*Also known as Nicotinamide

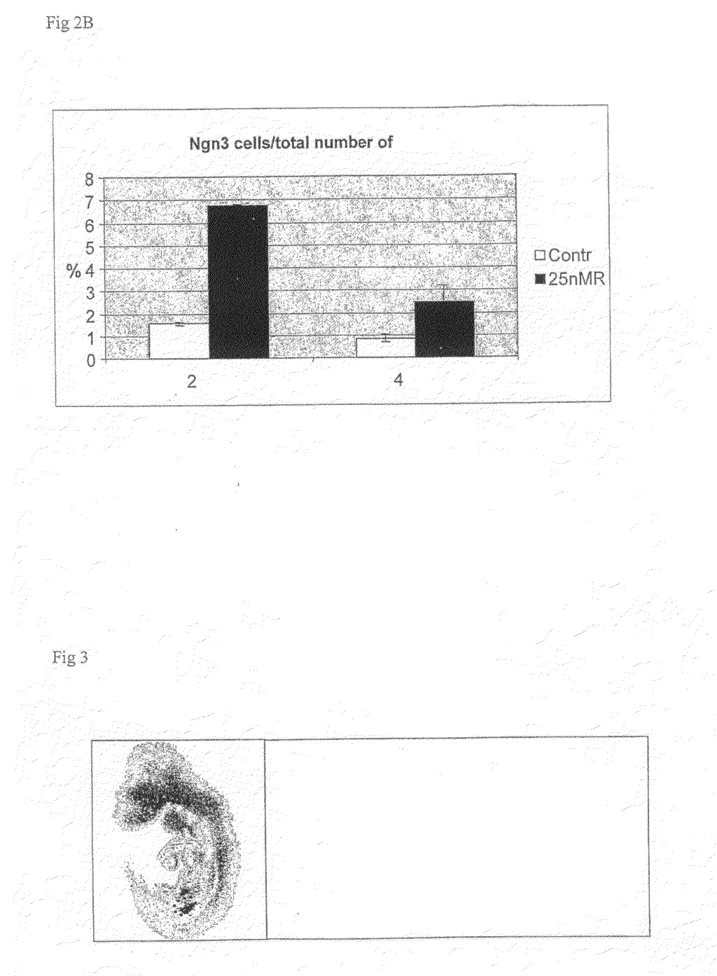

At this embryonic stage the dorsal pancreatic bud can be seen as a round structure forming from the gut endoderm that can be dissected out from the surrounding tissues (see FIG. 3). Using a mouth pipette individual isolated pancreatic buds were placed on 0.4 μm, pore 3,28 12 mm Millicell-CM PICM01250 filters (Millipore) that were subsequently placed in a twenty-four well plate (Costar) where each well contained 400 μl culture medium, consisting of DMEM-glutamax1 (Gibco), 1×N₂-supplement (Gibco) and PEST (Gibco).

Dulbecco's Modified Eagle Medium (D-MEM) (1×), liquid (Low Glucose) 21 885 with GlutaMAX™ I, 1000 mg/L D-Glucose, Sodium Pyruvate has the following characteristics: Recommended storage condition: +2° C. to +8° C.; Intended use(s): in vitro diagnostic IVD;

| Components | | mg/L |
|---|---|---|
| INORGANIC SALTS: | | |
| CaCI₂ × 2H₂O | 264.00 | |
| CaCI₂ (anhyd) | — | |
| Fe(NO₃)₃ × 9H2O | | 0.10 |
| KCI | | 400.00 |
| MgSO₄ (anhyd) | | — |
| MgSO₄ × 7H₂O | | 200.00 |
| NaCI | | 6400.00 |
| NaHCO₃ | | 3700.00 |
| NaH₂PO₄ × 2H₂O | | 141.00 |
| NaH₂PO₄ × H2O^{a} | | — |
| OTHER COMPONENTS: | | |
| D-Glucose | | 1000.00 |
| Phenol Red | | 15.00 |
| HEPES | | — |
| Sodium Pyruvate | | 110.00 |
| AMINO ACIDS: | | |
| L-Alanine | | — |
| L-Asparagine | — | |
| L-Arginine × HCI | | 84.00 |
| L-Aspartic Acid | | — |
| L-Cystine | | 48.00 |
| L-Cystine × 2HCI | | — |
| L-Glutamic Acid | | — |
| L-Glutamine | | — |
| L-Alanyl-L-Glutamine | | 862.00 |
| Glycyl-L-Glutamine | | — |
| Glycine | | 30.00 |
| L-Histidine HCI × H₂O | | 42.00 |
| L-Isoleucine | 105.00 | |
| L-Leucine | | 105.00 |
| L-Lysine × HCI | | 146.00 |
| L-Methionine | 30.00 | |
| L-Phenylalanine | | 66.00 |
| L-Proline | | — |
| L-Serine | | 42.00 |
| L-Threonine | 95.00 | |
| L-Tryptophan | 16.00 | |
| L-Tyrosine | | 72.00 |
| L-Tyrosine (disodium salt) | — | |
| L-Valine | | 94.00 |
| VITAMINS: | | |
| D-Ca pantothenate | | 4.00 |
| Choline Chloride | | 4.00 |
| Folic Acid | | 4.00 |
| i-Inositol | | 7.20 |
| Niacinamide* | 4.00 | |
| Pyridoxine HCI | | 4.00 |
| Pyridoxal HCI | | — |
| Riboflavin | | 0.40 |
| Thiamine HCI | 4.00 | |

^{a}Values shown are in conformance with the Tissue Culture Standards Committee, In Vitro 9:6
*Also known as Nicotinamide

N-2 Supplement, (100×) liquid represents a chemically defined system when used as a supplement to Neurobasal™. Allows for the maintenance of rat primary neurons in mass culture. Effective for the growth of most tumour cell lines of neuronal origin. Demonstrated growth of rat embryonic CNS progenitor cells. Recommended storage condition: dark, −5° C. to −25° C.

N-2 Supplement comprises

| Component | | μg/ml |
|---|---|---|
| Insulin | | 500.00 |
| Human transferring | | 10000.00 |
| Progesterone | 0.63 | |
| Putrascine | | 1611.00 |
| Selenite | | 0.52 |

Penicillin/Streptomycin Solution, liquid=PEST is 10,000 units/ml Penicillin and 10,000 μg/ml Streptomycin utilising penicillin G (sodium salt) and streptomycin sulphate: prepared in normal saline. Spectrum: Gram positive and gram negative bacteria. Recommended storage condition: −5° C. to −25° C.

The explants were cultured for six days in 37° C. and 5% CO₂ in culture medium alone (control explants) or with 25 nM all-trans retinoic acid (Sigma) added to the medium.

After six days of culture the explants were washed twice in PBS, fixated in 4% PFA in PBS for 25 minutes followed by two more washes in PBS. Using a small pair of scissors the filter was cut around the explant and the filter piece with the explant attached to it was placed in Tissue Tek O.C.T. Compound 4583 (Sakura). In the Tissue Tek the explant was gently removed from the filter using tungsten needles. A small drop of Tissue Tek was applied on a glass slide (Menzel) and the explant was placed in the drop using forceps and tungsten needles. The glass slides were put on dry ice to freeze the drops and were then stored in −80° C.

### Example 2

### Immunohistochemistry

The frozen explants were sectioned using a Cryostat into 8 μM thick sections that were collected on Superfrost Plus glass slides (Menzel). All explants from one experiment were put on the same slides. The slides were placed in a hybridisation chamber and blocked with blocking solution, TBST (50 mMT nsHCl, pH7.4, 150 mM MaCl, 0,1% Triton X-100)+10% FCS (fetal calf serum), for one hour. After blocking, primary antibodies diluted in fresh blocking solution were added and the slides were incubated overnight at 4° C. The following primary antibodies were used: guinea-pig α insulin (Linco) diluted 1/200, rabbit α glucagon (Euro Diagnostica) diluted 1/1000 and rabbit anti-ngn3 (raised against a GST-ngn3 fusion protein by AgriSeraAB) diluted 1/400. The slides were washed 3×5 minutes in TBST before incubation with secondary antibodies; a guinea-pig alexa488 (Molecular Probes) diluted 1/1000 and α rabbitCy3 (Jackson) diluted 1/400, for one hour in room temperature. The slides were then washed again 3×5 minutes in TBST before they were mounted using Vectashield with DAPI (Vector). The slides were analysed using a Nikon fluorescence microscope. The cells stained for insulin and glucagos respectively were counted and divided by the total number of cells that was counted using the DAPI staining which stains DNA i.e. every cell nucleus.

### Results

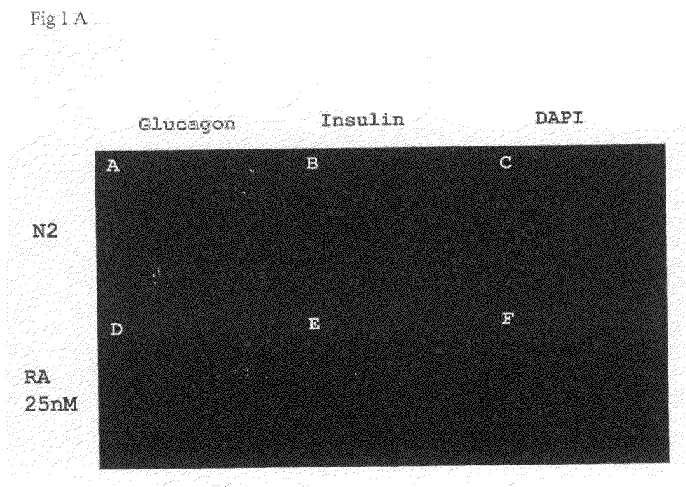

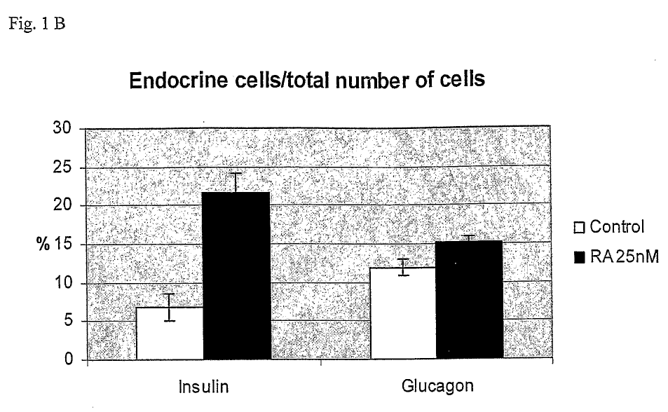

To investigate a role for RA in pancreatic cell differentiation mouse embryonic day (e) 10 dorsal pancreatic buds, which primarily contain pancreatic stem cells and very few differentiated pancreatic cells, were cultured in serum free media with or without RA for six days. The in vitro cultured explants were then scored for the appearance of endocrine cells expressing glucagon and insulin by immunohistochemistry using antibodies specific for glucagon and insulin. In vitro cultivation in media alone generated a few glucagon-(˜10% of total cells) and insulin-positive (˜5% of total cells) cells but the majority of cells were of non-endocrine origin (FIGS. 1 A-C and data not shown). In vitro culture of e10 pancreatic buds in media supplemented with all-trans RA (final concentration=25 nM) resulted in a more than 2-fold increase in endocrine cells (from ˜15% to ˜37%) as compared to explants cultured in media alone. Moreover, RA preferentially stimulated the differentiation of insulin-positive cells, which increased ˜4.5 fold, whereas glucagon-positive cells increased only ˜1,5 fold (FIG. 1D-F).

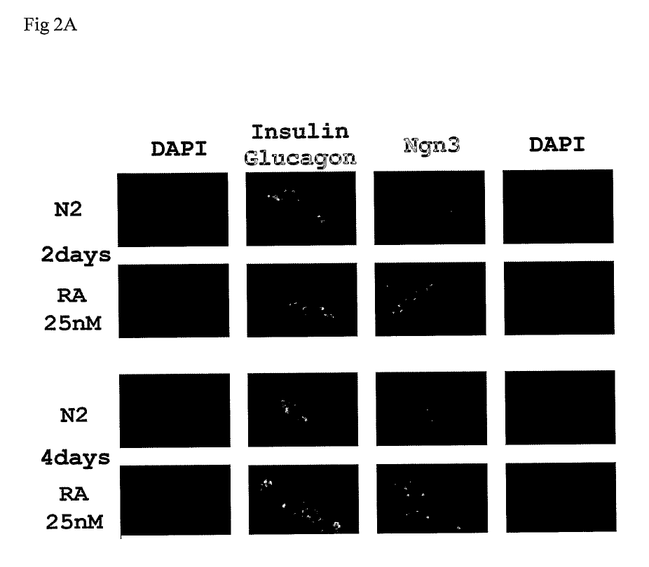

The RA-stimulated increased differentiation of glucagon- and insulin-positive cells was preceded by an increase in the amount of cells expressing the pro-endocrine factor neurogenin 3 (ngn3) that has been shown to promote pancreatic endocrine differentiation (Apelqvist et al., 1999) (FIG. 2).

Together these data provide evidence that RA-stimulate specification and differentiation of pancreatic stem cells into endocrine, and in particular insulin-producing, cells. These data also suggest that RA-antagonists would block the differentiation of pancreatic stem cells into endocrine and insulin producing β-cells. Consequently, RA or analogues thereof and RA-agonists holds a huge potential as stimulators for the differentiation, in vitro and in vivo, of stem and progenitor cells into pancreatic endocrine and insulin producing β-cells, which in turn can be used for therapeutical transplantation and/or intervention treatment of type 1 and type 2 diabetes.

### REFERENCES

Apelqvist Å., Li, H., Sommer L. Notch-signalling controls pancreatic cell differentiation. Nature 400, 877-881, 1999.
Diez del Corral R, Olivera-Martinez I, Goriely A, Gale E, Maden M, Storey K. Opposing FGF and retinoid pathways control ventral neural pattern, neuronal differentiation, and segmentation during body axis extension. Neuron 40, 65-79, 2003.
Edlund, H. Pancreatic organogenesis-developmental mechanisms and implications for therapy. Nat. Rev. Genet. 3, 524-532, 2002.
Hiroyuki Kagechika, Koichi Shudo, Synthetic Retinoids: Recent developments concerning structure and clinical utility Journal of medicinal chemistry, 2005, Vol. 48, No. 19.
Hart, A., Papadopolous, S., and Edlund H. Fgf10 maintains Notch activation, stimulates proliferation and blocks differentiation of pancreatic progenitor cells. Dev. Dyn. 228:185-193, 2003.
Novitch B G, Wichterle H, Jessell T M, Sockanathan S. A requirement for retinoic acid-mediated transcriptional activation in ventral neural patterning and motor neuron specification. Neuron 40, 81-95, 2003.

1.-12. (canceled)

13. A method of forming pancreatic hormone-producing endocrine cells in vitro, wherein embryonic pancreatic stem cells are treated with one or more retinoids and/or retinoic acid at a concentration of 10-200 nM in vitro and cultured.

14. The method according to claim 13, wherein the pancreatic stem cells are chosen from the dorsal pancreatic bud.

15. The method according to claim 13, wherein the pancreatic stem cells are obtained from six to thirteen-day-old embryos.

16. The method according to claim 13, wherein the pancreatic stem cells are obtained from eight to twelve-day-old embryos.

17. The method according to claim 13, wherein the pancreatic stem cells derive from vertebrates such as from rodents such as mice, rats; pigs; monkeys and humans.

18. The method according to claim 13, wherein the treatment with at least one of the group consisting of retinoids and retinoic acid is conducted at a concentration 10-200 nM.

19. The method according to claim 13, wherein the treatment with retinoids and/or retinoic acid is performed during 2 to 10 days, such as 3 to 8 days, especially 5-7 days at 30° C.-40° C.₅ preferably at 35° C.-39° C. most preferred at 37° C.

20. The method according to claim 13, wherein the retinoids and/or retinoic acid are chosen from retinoic acid and isomers thereof such as all-trans-retinoic acid, and 9-cis-retinoic acid, preferably all-trans-retinoic acid.

21. Pancreatic hormone-producing endocrine cells, wherein said cells can be obtained by the method of forming pancreatic hormone-producing endocrine cells in vitro wherein pancreatic stem cells are treated with one or more retinoids and/or retinoic acid in vitro and cultured.

22. Use of pancreatic hormone-producing endocrine cells, obtainable by treating pancreatic stem cells with one or more retinoids and/or retinoic acid in vitro and culturing them, for the production of a pharmaceutical composition for transplantation and/or treatment of type 1 and type 2 diabetes.

23. A method for treatment of type 1 and type 2 diabetes wherein pancreatic hormone-producing endocrine cells, obtainable by treating pancreatic stem cells with one or more retinoids and/or retinoic acid in vitro and culturing them are transplanted into an individual in need thereof.

24. Use of one or more retinoids and/or retinoic acid for the differentiation, in vitro of pancreatic stem cells into pancreatic hormone-producing endocrine cells, such as glucagon producing cells, and in particular, insulin producing β-cells.

25. The method according to claim 14, wherein the pancreatic stem cells are obtained from six to thirteen-day-old embryos.

26. The method according to claim 14, wherein the pancreatic stem cells are obtained from eight to twelve-day-old embryos.

27. The method according to claim 25, wherein the pancreatic stem cells are obtained from eight to twelve-day-old embryos.

28. The method according to claim 27, wherein the pancreatic stem cells derive from vertebrates such as from rodents such as mice, rats; pigs; monkeys and humans.

29. The method according to claim 26, wherein the pancreatic stem cells derive from vertebrates such as from rodents such as mice, rats; pigs; monkeys and humans.

30. The method according to claim 29, wherein the treatment with retinoids and/or retinoic acid is conducted at a concentration 10-200 nM.

31. The method according to claim 25, wherein the treatment with retinoids and/or retinoic acid is conducted at a concentration 10-200 nM.
